(19) United States
(12) Patent Application Publication
(10) Pub. No.: US 2010/0022789 A1
(43) Pub. Date: Jan. 28, 2010

(54) Catalytic compositions for the metathesis of unsaturated fatty bodies with olefins and metathesis methods using catalytic compositions

(75) Inventors: Gérard Mignani, Lyon (FR); Virginie Pevere, Lyon (FR); Hélène Olivier-Bourbigou, Saint Genis Laval (FR); Christophe Vallee, Sassenage (FR); Mikael Berthod, Lyon (FR); Cécilia Citadelle, Lyon (FR)

Correspondence Address: BUCHANAN, INGERSOLL & ROONEY PC, POST OFFICE BOX 1404, ALEXANDRIA, VA, 22313-1404, US

(73) Assignees: RHODIA OPERATIONS, Aubervilliers (FR); IFP, Rueil-Malmaison (FR)

(21) Appl. No.: 12/458,856

(22) Filed: Jul. 24, 2009

(30) Foreign Application Priority Data
Jul. 25, 2008 (FR) 0855134

(51) Int. Cl. C07C 67/00; C07F 15/00; C07C 51/00

(52) U.S. Cl. 554/161; 548/403; 554/124

(57) ABSTRACT

Catalytic compositions useful for carrying out the metathesis of unsaturated fatty bodies, in particular unsaturated fatty bodies comprising oleic acid or oleic acid esters, contain a ruthenium-based catalyst which can be isolated and which has the formula (I) below:

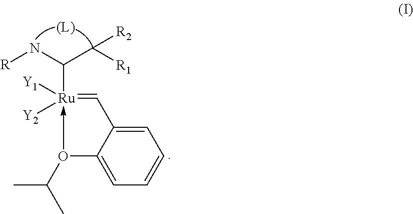

### CROSS-REFERENCE TO PRIORITY APPLICATION

This application claims priority under 35 U.S.C. § 119 of FR 0855134, filed Jul. 25, 2008, hereby expressly incorporated by reference and each assigned to the assignees hereof.

### BACKGROUND OF THE INVENTION

1. Technical Field of the Invention

The present invention relates to catalytic compositions which are very suitable, inter alia, for the metathesis of olefins. More precisely, the catalytic compositions of the invention are found to be particularly advantageous for carrying out metathesis reactions which involve unsaturated fatty bodies, especially fatty esters of the type obtained from vegetable or animal oils.

2 Description of Background and/or Related and/or Prior Art

The metathesis of olefins has seen a significant development over recent years in fields as varied as petrochemistry, polymers, oleochemistry and fine chemistry and has developed in particular as an important tool in the formation of carbon-carbon bonds.

The reaction which is referred to as metathesis is a well known reaction which occurs from two olefin compounds (carrier compounds of C═C functions). This reaction leads to an exchange of alkylidenes of the two initial olefin compounds, in accordance with the reaction below:

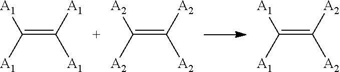

A metathesis reaction of this type may involve two different olefin compounds, which is referred to as a “cross-metathesis”.

On the other hand, a metathesis reaction may, involve only one olefin compound which reacts with itself, which is referred to as a “homo-metathesis”. Such a homo-metathesis typically allows the synthesis of symmetrical olefins from an asymmetric olefin, typically in accordance with the following reaction:

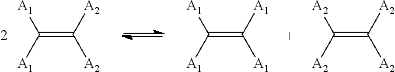

There are a very large number of cross-metathesis and homo-metathesis reactions which are generally carried out in the presence of catalysts which are derived from transition metals, typically based on transition metals from the groups 4 to 10, either in a homogeneous phase or in a heterogeneous phase. In this context, it is well known that the nature of the catalyst which can be used to carry out a specific metathesis reaction is intended to be adapted on a case by case basis in particular in accordance with the precise nature of the olefin compounds involved.

The present invention is associated more specifically with metathesis reactions which specifically involve olefin compounds such as unsaturated fatty body type.

In the context of the present description, the term “unsaturated fatty body” is intended to refer to a compound which carries an unsaturated fatty chain, this fatty chain being a linear or branched hydrocarbon chain (preferably linear) comprising at least a double C═C bond (typically a linear or branched alkenyl chain), and typically containing from 10 to 22 atoms of carbon. The notion of “unsaturated fatty body” in the context of the present description includes in particular the unsaturated compounds present in vegetable and animal oils and the esters of fatty acids contained in these oils as well as the corresponding fatty acids (non-esterefied carboxylic acids).

There is very little teaching relating to suitable catalysts when the metathesis reaction specifically involves an unsaturated fatty body of this type as an olefin compound.

In this context, it is possible to mention the article by J. C. Mol (Topics in Catalysis, 2004, 27, 1) which reviews the catalytic conversions which are available for converting fatty acid esters by using metathesis reactions.

Furthermore, WO 2008/01096 describes catalysts based on ruthenium or osmium which are complexed by ligands which include an N-heterocyclic carbene (NHC) and sets out that they are suitable for carrying out a specific metathesis reaction, referred to as ethenolysis, which involves reacting an olefin with ethylene CH2═CH2 which allows the synthesis of terminal olefins (that is to say, which terminate in a terminal group>C═CH2). In WO 2008/010961, the ethenolysis reaction is used to carry out the conversion of “seed oils” in the presence of ethylene.

One aim of the present invention is to provide new catalytic compositions which are well suited to catalyzing in an effective manner, metathesis reactions which involve unsaturated fatty bodies as olefin compounds. In this context, the invention is especially intended to provide catalytic compositions which can preferably be used both to catalyze cross-metathesis reactions involving the reactions of unsaturated fatty bodies with olefin compounds and homo-metathesis reactions of unsaturated fatty bodies.

### SUMMARY OF THE INVENTION

To this end, the present invention features catalytic compositions which comprise (and optionally is constituted by) a ruthenium-based catalyst having the formula (I) below:

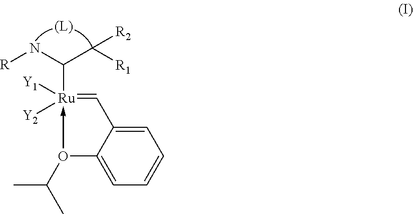

wherein:

Y₁ and Y₂ are two anionic ligands which may be identical or different;

R is a branched alkyl radical, an aryl radical or a substituted aryl radical;

R₁ is a group comprising an aromatic radical, R₁ typically being an aryl, alkylaryl or arylalkyl radical which may or may not be substituted;

R₂ is an aryl, alkylaryl or arylalkyl radical which may or may not be substituted and which is identical or different to R₁; or an alkyl radical; and

the group -(L)- is a divalent hydrocarbon radical, with the proviso that the ring having the following formula:

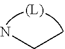

formed by this divalent radical -(L)-, nitrogen, the carbon linked to the ruthenium and the carrier carbon of the radicals R₁ and R₂, is a ring having 5 or 6 atoms, the nitrogen atom included.

According to a specific aspect, the invention also relates to catalysts which comply with the formula (I) mentioned above, which are compounds which can be isolated as such.

In the context of the present invention, the work of the inventors has shown that ruthenium-based catalysts which comply with the above formula (I), and the catalytic compositions comprising it, allow metathesis reactions to be carried out for unsaturated fatty bodies (in particular fatty acid esters) with olefin and homo-metathesis reactions of unsaturated fatty bodies, with high levels of conversion and significant selectivity in terms of the products sought.

Furthermore, the catalytic compositions of the invention have the advantage of not catalyzing the isomerization reactions of the double olefin bonds of the products and the reagents used, which allows the above-mentioned metathesis reactions to be carried out without the formation of undesirable isomer by-products.

Furthermore, the catalytic compositions of the invention have been found to be simple to prepare, the inventors having developed a method for preparing these compounds having the formula (I) which comprises a limited number of steps and which is found to be effective and inexpensive, which allows its implementation on an industrial scale to be envisaged, particularly since it involves precursors which are relatively inexpensive and available. This method for preparing the compositions of the invention, which will be described in detail below, constitutes another specific object of the present invention.

These various aspects of the invention will now be described in greater detail.

### DETAILED DESCRIPTION OF BEST MODE AND SPECIFIC/PREFERRED EMBODIMENTS OF THE INVENTION

The catalytic compositions and the catalyst having the formula (I):

The catalytic compositions of the invention contain a catalyst which complies with the above-mentioned formula (I).

In this catalyst having the formula (I), the anionic ligands Y₁ and Y₂ are identical or different (and generally identical) and may typically be selected from halides, alkyls, aryls, sulfate, alkylsulfates, arylsulfates, alkylsulfonates arylsulfonates, alkylsulfinates, arylsulfinates, acyls, carbonates, carboxylates, alcoholates, phenates, amides and pyrolides. These ligands may be substituted in particular by one or more of the following substituting groups: C₁-C₁₂ alkyl, C₁-C₁₂ alcoholates, C₅-C₂₄ aryl or halides. These substituting groups, with the exception of the halides, may themselves be substituted in particular by one or more of the following groups: halides, C₁-C₆ alkyls, C₁-C₆ alcoholates, and aryls.

Preferably, the ligands Y₁ and Y₂ are anionic ligands which are selected from the following ligands: halides (chlorides or bromides in particular), benzoate, CF₃CO₂, CH₃CO₂, (CH₃)₃CO, (CF₃)₂(CH₃)CO, (CF₃)(CH₃)₂CO, C₆F₅O, PhO, MeO, EtO, tosylate, mesylate or trifluoromethane-sulfonate, or pyrolide.

According to a specific embodiment, the ligands Y₁ and Y₂ may be connected together within the same species which has the character of a double anionic ligand (typically a bidentate ligand). By way of example of such a species comprising two connected ligands Y₁ and Y₂, it is possible to mention in particular C₆H₄O₂.

According to a particularly preferred embodiment, the two ligands Y₁ and Y₂ are chlorides.

Furthermore, in the catalyst having the formula (I) which is present in the catalytic compositions of the invention, the divalent group -(L)- preferably complies with the following formula:

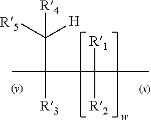

wherein:

(x) and (y) indicate the two bonds which are established from the carbon atom which carries the groups R₁ and R₂ and the nitrogen atom which carries the group R respectively in the compound having the formula (I),

w is a number equal to 1 or 2; and

each of the groups R′₁, R′₂, R′₃, R′₄ and R′₅, which may be identical or different, is an atom of hydrogen or an alkyl, cycloalkyl, aryl or arylalkyl group.

Preferably, the catalyst present in the catalytic composition of the invention complies with the following formula (II):

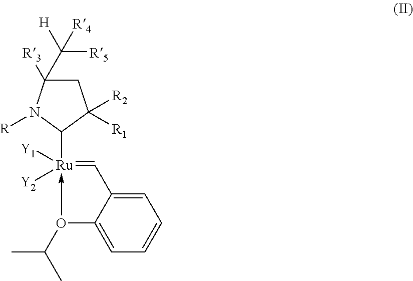

wherein:

R is a branched alkyl group, an aryl group, or a substituted aryl group;

R₁ is an aryl or substituted aryl group;

R₂ is an aryl group or a substituted aryl group which may be identical or different to R₁, or an alkyl group;

R′₃ is a hydrogen atom, an alkyl group, an aryl group, a substituted aryl group;

each of R′₄ and R′₅, which may be identical or different, is an atom of hydrogen, an alkyl group, an aryl group, or a substituted aryl group.

Catalysts which are particularly advantageous according to the invention are those which comply with the above formula (II), and in which:

R is a tert-butyl group; a phenyl group which is substituted by 3 methyl or ethyl groups in the position o, o′ and p; a phenyl group which is substituted by 2 isopropyl or tert-butyl groups in the position o and o′; a phenyl group which is substituted by 3 isopropyl or tert-butyl groups in the position o, o′ and p; or a phenyl group which is substituted by two ethyl groups in the positions o and o′;

R₁ is a phenyl, substituted phenyl, naphthyl or substituted naphthyl group;

R₂ is a phenyl, substituted phenyl, naphthyl or substituted naphthyl group, which may be identical or different to R₁, or a methyl, ethyl, propyl or isopropyl group,

R′₃ is an atom of hydrogen or a methyl group,

each of the groups R′₄, R′₅, which may be identical or different, is an atom of hydrogen or a methyl group.

Catalysts which are even more preferred are those which comply with the above-mentioned formula (II) and in which:

R is a phenyl group which is substituted by two isopropyl groups in the position o and o′; a phenyl group which is substituted by 3 methyl or isopropyl groups in the position o, o′ and p; or a phenyl group which is substituted by two ethyl groups in the positions o and o′ or a tert-butyl group;

R₁, R₂, which may be identical or different, are selected from a phenyl, substituted phenyl, naphthyl or substituted naphthyl group;

R′₃ is a methyl group,

R′₄ and R′₅ are both atoms of hydrogen.

Catalysts which are particularly advantageous according to the invention comply with the formula (III) below (which comprises two aryl groups):

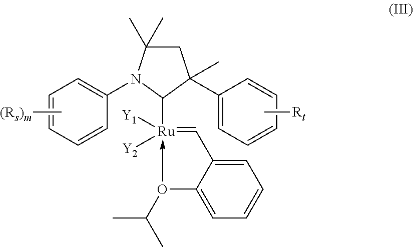

wherein:

m is the number of substituents carried by the aryl group which is bonded to the nitrogen, this number being equal to 1, 2 or 3, m being more preferably equal to 2 or 3,

each of the m groups Rₛ, which may be identical or different, is a substituent selected from the alkyl or alkoxy groups which have from 1 to 4 atoms of carbon,

Rₜ is one or more substituents, which may be identical or different, selected from the alkyl or alkoxy groups which have from 1 to 4 atoms of carbon; and

Y₁ and Y₂ have the above-mentioned meanings.

When m is equal to 2, the substituents are preferably in the position ortho and ortho′ relative to the carbon atom which is bonded to the nitrogen atom, and when m is equal to 3, the substituents are advantageously in the position ortho, ortho′ and para relative to the carbon atom which is bonded to the nitrogen atom.

Catalysts which are advantageous according to the invention are in particular the compounds which comply with one of the formulae (IIIa) to (IIIc) below:

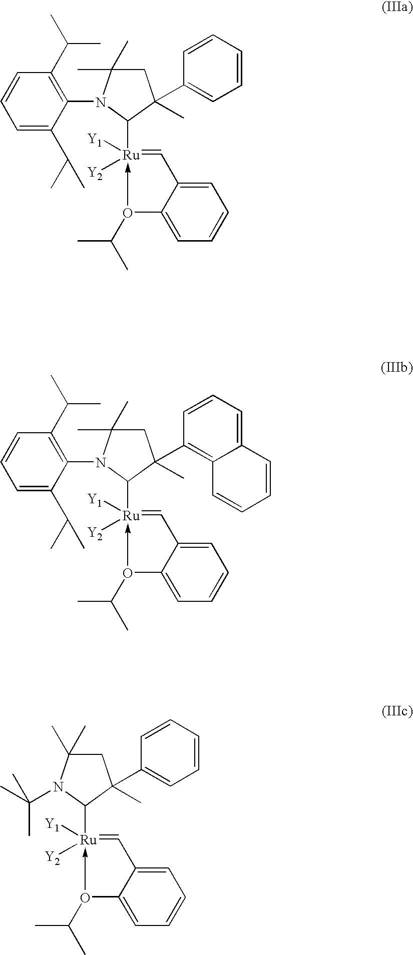

wherein:

Y₁ and Y₂ have the above-mentioned meanings.

In the context of the present description, the term “alkyl” is intended to refer to a hydrocarbon chain which is linear or branched at C₁-C₁₅, preferably at C₁-C₁₀ and even more preferably at C₁ to C₄. Examples of preferred alkyl groups are in particular methyl, ethyl, propyl, isopropyl, butyl, isobutyl, t-butyl.

The term “alkoxy” is intended to refer to an alkyl-O— group in which the term alkyl has the meaning given above. Preferred examples of alkoxy groups are the methoxy or ethoxy groups.

The term “alkoxycarbonyl” refers to the group alkoxy-C(O)— in which the alkoxy group has the definition given above.

The term “alkenyl” is intended to refer to a linear or branched hydrocarbon chain which comprises a double bond at C₂-C₈, preferably at C₂-C₆, and more preferably at C₂-C₄. Examples of preferred alkenyl groups are in particular the groups vinyl, 1 -propenyl, 2-propenyl, isopropenyl, 1-butenyl, 2-butenyl, 3-butenyl and isobutenyl.

The term “alkynyl” is intended to refer to a linear or branched hydrocarbon chain which comprises a triple bond at C₂-C₈, preferably at C₂-C₆, and more preferably at C₂-C₄. Examples of preferred alkynyl groups are in particular the groups ethynyl, 1 -propynyl, 1 -butynyl, 2-butynyl.

The terms “alkenyloxy” and “alkynyloxy” are intended to refer respectively to an alkenyl-O and alkynyl-O group in which the terms alkenyl and alkynyl have the meaning given above.

The term “cycloalkyl” is intended to refer to a hydrocarbon group which is cyclic or monocyclic, preferably at C₃-C₁₀, a cyclopentyl or cyclohexyl group which is polycyclic (bi- or tricyclic) at C₄-C₁₈, in particular adamantyl or norbornyl.

The term “aryl” is intended to refer to an aromatic mono- or polycyclic group, preferably mono- or bi-cyclic at C₆-C₂₀, preferably phenyl or naphthyl. When the group is polycyclic, that is to say, it comprises more than one cyclic core, the cyclic cores can be condensed in pairs or attached in pairs by σ bonds. Examples of (C₆-C₁₈) aryl groups are in particular phenyl, naphthyl.

The term “aryloxy” is intended to refer to an aryl-O group in which the aryl group has the meaning given above.

The term “arylalkyl” or “aralkyl” is intended to refer to a linear or branched hydrocarbon group which carries a monocyclic aromatic ring at C₇-C₁₂, preferably benzyl: the aliphatic chain comprising 1 or 2 atoms of carbon.

It should be noted that, as soon as one of the above-mentioned groups R₁, R₂, R′₁, R′₂, R′₃, R′₄, R′₅ comprises a ring, it can be substituted by at least one substituent (preferably two or three). As preferred examples of substituents, it is possible to cite in particular the groups alkyl, alkoxy, alkenyl, alkynyl, cycloalkyl, aryl, arylalkyl, amino, amino substituted by alkyl, cycloalkyl groups; a nitrile group; an atom of halogen, preferably chlorine or fluorine; a haloalkyl group, preferably perfluoromethyl.

Preparation of the catalytic compositions of the invention:

The catalytic compositions of the present invention which comprise (and optionally consist of) the above-mentioned catalysts may advantageously be obtained in accordance with a method which comprises a step in which there are placed in contact:

a ruthenium-based precursor which complies with the general formula (IV) below:

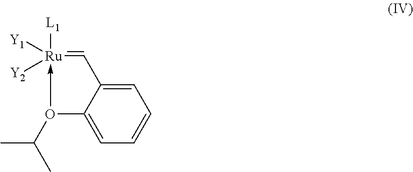

wherein:

Y₁ and Y₂ are ligands, which may be identical or different, as defined above for the formula (I); and

L₁ is a neutral electron-donating ligand; and

a carbene which complies with the formula (V) below:

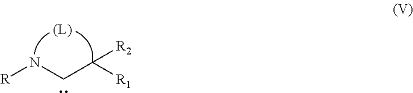

wherein:

R, R₁, R₂ and -(L)- are as defined above for the above-mentioned formula (I).

In the method for preparing the catalytic compositions of the present invention, the step for placing the compounds (IV) and (V) in contact may advantageously be followed by a step for isolating the catalyst formed by the reaction of the compounds (IV) and (V) and/or by a step for adding a solvent into the composition obtained following the reaction of the compounds (IV) and (V) or after isolation of the catalyst formed.

Preferably, in the precursor (IV) used in the present preferred description, Y₁ and Y₂ are halides, benzoate, CF₃CO₂, CH₃CO₂, (CH₃)₃CO, (CF₃)₂(CH₃)CO, (CF₃)(CH₃)₂CO, C₆F₅O, PhO, MeO, EtO, tosylate, mesylate or trifluoromethane-sulfonate or pyrolide. More preferably, Y₁ and Y₂ are chlorides.

Furthermore, in the precursor (IV), the ligand L1 which is neutral and which donates electrons is advantageously selected from phosphorus-containing ligands, such as phosphines, phosphites, phosphinites, phosphonites; or arsines, stibines, nitrogen-containing ligands, such as amines, which may or may not be cyclic and which may or may not be aromatic, amides, imines.

More preferably, the ligand L₁ is a phosphine group typically having the formula PR₃R₄R₅, where R₃, R₄ and R₅ are preferably alkyl, cycloalkyl or aryl groups. Advantageously, the ligand L₁ present in the compound (IV) is a phosphine group selected from the groups P(cyclohexyl)₃, P(cyclopentyl)₃; P(isopropyl)₃; or P(phenyl)₃.

In this manner, by way of example of a compound (IV) which is advantageous according to the invention, it is possible to mention, for example, the compound having the following formula (IVa):

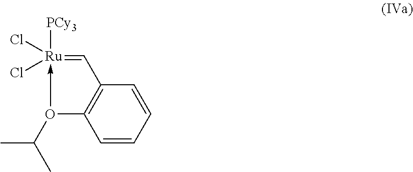

wherein:

Cy designates a cyclohexyl group.

The carbene (V) which is used in the method for preparing catalytic compositions of the present invention is itself a carbene having an alkylaminocyclic structure (referred to as “CAAC”; an abbreviation for “Cyclic Alkyl Amino Carbene”.) Carbenes of this type are compounds which are known, in particular from WO2006/138166.

The carbene (V) which is used in the context of the present invention is a specific CAAC carbene, which specifically has an aryl or substituted aryl group as an R₁ group. The synthesis thereof presents no specific difficulty and it can be prepared according to any method known per se.

According to an advantageous embodiment, the carbene (V) used in the preparation of the catalytic compositions of the invention is prepared by reacting a base over a corresponding iminium salt. In this instance, typically, the carbene (V) is obtained by reacting:

an iminium salt which complies with the formula (VI) below:

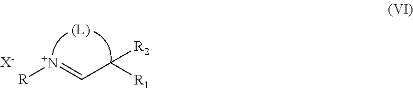

wherein:

R, R₁, R₂ and -(L)- have the above-mentioned definitions; and

X⁻ is an anion, preferably selected from a halide, an acetate group, a trifluoroacetate, a mesylate or a tosylate or a mixture of these ions, this anion X⁻ more advantageously being a chloride or a bromide and even more advantageously a chloride which may in particular be in the form Cl⁻, or HCl₂⁻; and

a basic, organic or inorganic compound.

Carbenes having the formula (V) may advantageously be prepared by reacting precursors of the iminium salt type having the formula (VI) with a strong base, for example, butyllithium, sodium amide or potassium bis(trimethylsilyl)amide (KHMDS), in an aprotic organic solvent, for example, THF, under anhydrous conditions.

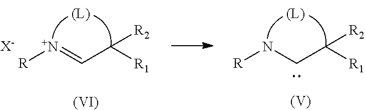

Regardless of the exact method of implementation, the formation of the carbene from iminium salt may take place either upstream of the reaction which places the carbene and the compound (IV) in contact, or in situ in the reaction medium from the carbene and the compound (IV).

When it is desirable to prepare according to the invention compositions comprising catalysts which comply with the above-mentioned formula (II), the carbene (V) used advantageously complies with the formula (V.2) below:

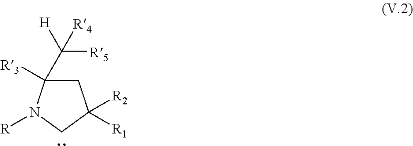

wherein:

R, R₁, R₂, R′₃, R′₄ and R′₅ have the above-mentioned meanings.

This carbene (V.2) may typically be obtained by a basic attack of an iminium salt having the formula (VI.2) below:

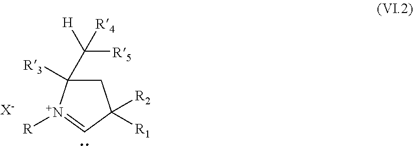

wherein:

R, R₁, R₂, R′₃, R′₄ and R′₅ have the above-mentioned meanings; and X⁻ is an anion of the above-mentioned type, advantageously a chloride, in particular in the form Cl⁻, and/or HCl₂⁻ (and generally at least partially in the form of HCl₂⁻).

In the same manner, when it is desirable to prepare according to the invention compositions comprising catalysts which comply with the formula (III) mentioned above, the carbene (V) used advantageously complies with the formula (V.3) below:

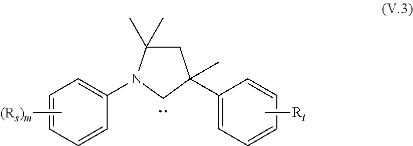

wherein:

m, Rs and Rt have the above-mentioned meanings.

This carbene (V.3) can typically be obtained by a basic attack of an iminium salt which complies with the formula (VI.3) below:

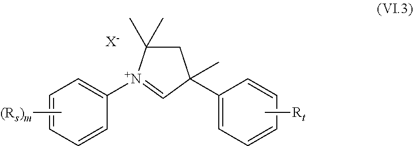

wherein:

m, Rs and Rt have the above-mentioned meanings; and X⁻ is an anion of the above-mentioned type, advantageously a chloride, in particular in the form Cl⁻ or HCl₂⁻.

Carbenes (V) which are advantageous for the implementation of the method of the invention are the following carbenes:

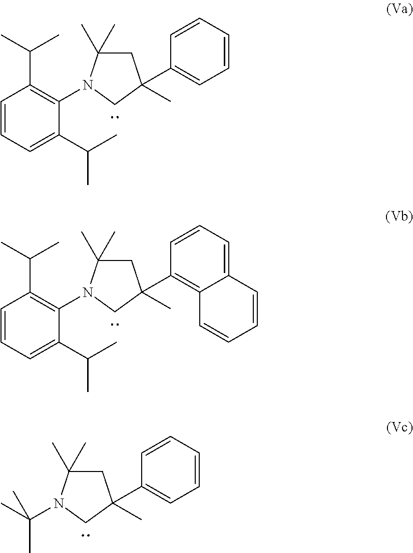

which allow, respectively, access to the catalysts having the formula (IIIa), (IIIb) and (IIIc) described above in the present description. These specific carbenes can again be synthesized from corresponding iminium salts.

Iminium salts having the formula (VI) which are used as a precursor of carbenes (V) can be synthesized in accordance with any method known per se. According to an advantageous embodiment, the iminium salts (VI) are prepared according to a method comprising the following steps:

reaction of an unsaturated aldehyde having the formula (C) with an amine having the formula (D) to form an imine having the formula (E), preferably in the presence of a strong protic acid as a catalyst, for example, methanesulfonic acid or p-toluenesulfonic acid (PTSA), and optionally in the presence of an organic solvent, for example, toluene, in accordance with the following reaction:

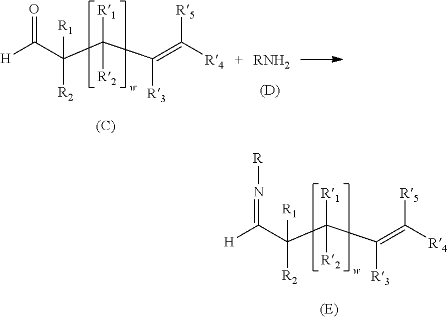

wherein:

R, R₁, R₂, R′₁,R′₂, R′₃, R′₄, R′₅ and w have the above-mentioned meanings; then

reaction of imine having the formula (E) with a strong protic acid, advantageously hydrochloric acid (typically HCl in gaseous form), followed by heating the reaction medium to a temperature greater than or equal to 60° C., whereby there is obtained, by cyclisation, the iminium salt having the formula (VI), in accordance with the reaction below:

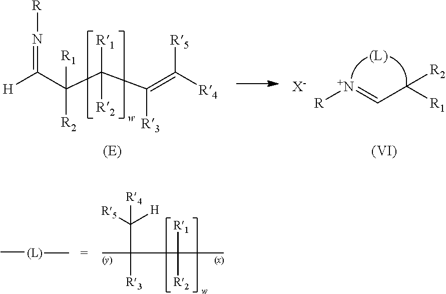

wherein:

R, R₁, R₂, R′₁, R′₂, R′₃, R′₄, R′₅, w, (x), (y) and -(L)- have the above-mentioned meanings.

In the step of reacting unsaturated aldehyde having the formula (C) with the amine having the formula (D) to form the imine having the formula (E), the conversion of the reaction is generally improved by eliminating water from the reaction admixture, for example, by means of azeotropic distillation or by adding a dehydrating agent, such as a molecular sieve, to the reaction medium.

The unsaturated aldehyde having the formula (C) above can advantageously be obtained by reacting an aldehyde having the formula (A) with an unsaturated reagent having the formula (B) which carries a leaving group Y, for example, chlorine or bromide, in the presence of a base, for example, sodium hydroxide, and a phase transfer catalyst, for example, tetrabutylammonium bromide, in a biphase admixture which comprises an organic solvent, for example, toluene, and an aqueous phase, in accordance with the following reaction:

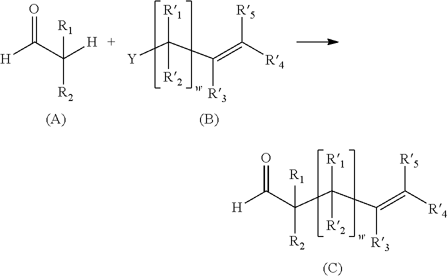

where R, R₁, R₂, R′₁, R′₂, R′₃, R′₄, R′₅ and w have the above-mentioned meanings.

In accordance with a particularly advantageous embodiment, the method for preparing the catalytic composition of the invention comprises the following steps:

(E1) at least one iminium salt which complies with the above-mentioned formula (VI) is placed in contact with at least one base, which may be organic or inorganic, whereby a carbene having the formula (V) is formed;

(E2) optionally, the carbene (V) formed during step (E1) is isolated;

(E3) at least one ruthenium-based precursor generally having the formula (IV) is added to the carbene obtained in step (E1) and is optionally isolated beforehand in step (E2), whereby the catalyst having the formula (I) is formed;

(E4) optionally, the catalyst formed in step (E3) is isolated,

(E5) optionally, a solvent is added to the catalyst formed in step E3, optionally isolated beforehand in step (E4).

Steps (E1) to (E5) are advantageously carried out under the following conditions:

Step (E1): placing the iminium salt in contact with a base:

In step (E1), placing the compound (VI) in contact with the base is advantageously carried out at a temperature of from −78° C. and +150° C., preferably from 0 and 80° C.

The duration of the reaction is generally from 30 minutes and 15 hours, preferably from 2 hours and 5 hours.

Step (E1) is typically carried out in a solvent medium, the solvents used for this step being selected from conventional polar or apolar organic solvents, such as aromatic or aliphatic hydrocarbons, such as toluene, xylene, cyclohexane, chlorine-containing solvents such as dichloromethane, ethers such as tetrahydrofuran or diethylether, or dioxane. These solvents can be used alone or in admixture. These solvents are preferably dried, by means of distillation or by being passed over an adsorbent, before being used.

The concentration of compound (VI) and base may vary to quite a wide degree in step (E1), typically from 0.01 and 10 mol/L, preferably from 0.05 and 1 mol/L.

Furthermore, the molar ratio of the quantity of base to the quantity of iminium salt (VI) introduced in step (E1) is generally from 1 and 10 and preferably from 1 and 5.

Optional step (E2): isolation of carbene (V) formed in step (E1):

The solution obtained following step (E1) may be used directly in step (E3). However, it may be found to be advantageous in some cases to isolate the carbene (V) formed in step (E1).

If necessary, it is possible to isolate the carbene (V), for example, by evaporating the solvent of step (E1) then optionally by extracting the solid obtained using an appropriate solvent, then finally by evaporating this solvent. The extraction solvent used for this step is advantageously selected from aliphatic hydrocarbons, such as pentane, hexane, heptane or cyclohexane and admixtures of these solvents. These solvents are preferably dried, by means of distillation or passage over an adsorbent, before being used.

Step (E3): adding the ruthenium compound to the carbene (V):

In step (E3), the precursor of ruthenium having the formula (IV) is added to the medium obtained following step (E1), or, if necessary, following step (E2).

The reaction of step (E2) can be carried out at a temperature of from −20 and 150° C., and preferably from 10 and 40° C.

The duration of the reaction is itself generally in the order of from 10 minutes to 12 hours and it is preferably from 30 minutes and 6 hours.

When the carbene (V) has been isolated in step (E2), it is typically found to be preferable to further add a solvent to the reaction medium, in particular to optimize the reaction from the compounds (IV) and (V). More generally, step (E3) may advantageously be carried out in a solvent medium. If necessary, the solvents which can be used in step (E3) may be the same as those recommended for step (E1). In this manner, when a solvent is used in step (E3), it may, for example, be selected from conventional polar or apolar organic solvents, such as aromatic or aliphatic hydrocarbons such as toluene, xylene, cyclohexane, chlorine-containing solvents such as dichloromethane, ethers such as tetrahydrofuran or diethylether and admixtures of these solvents. These solvents are preferably dried by means of distillation or by being passed over an adsorbent, before being used.

The concentration of the reagents in step (E3) may vary to a very wide degree, typically from 0.01 and 10 mol/L, the concentrations preferably being from 0.05 and 1 mol/L.

Furthermore, it is preferable for the molar ratio from the quantity of iminium salt introduced in step (E1) to the quantity of ruthenium precursor introduced in step (E3) to be from 1 and 10, more advantageously from 1 and 3.

Optional step (E4): isolation of the catalyst (I) formed in step (E3):

Optionally, the catalyst formed in step (E3) can be isolated. This isolation typically involves evaporation of the solvent used in step (E3), then optionally one or more washing operations of the solid obtained by means of an appropriate solvent, then filtration. The isolated product is thus obtained in the form of a solid.

If necessary, the washing solvent used for this step is preferably selected from aliphatic hydrocarbons, such as pentane, hexane, heptane or cyclohexane. These solvents are preferably dried by means of distillation or by being passed over an adsorbent, before being used.

Optional step (E5): Addition of a solvent:

Before using the catalytic composition, it is possible to dissolve it in a solvent, in particular to fluidize it.

If necessary, the solvent is generally added to obtain a concentration of catalyst in the diluted composition ranging from 0.001 to 10 mol/L, for example, from 0.005 and 1 mol/L.

If necessary, the solvent used to carry out the fluidification of the catalytic composition in step (E5) is preferably selected from conventional organic polar or apolar solvents, such as aromatic or aliphatic hydrocarbons such as toluene, xylene, cyclohexane, chlorine-containing solvents such as dichloromethane, ethers such as tetrahydrofuran or diethylether and admixtures of these solvents. Before being used, these solvents are preferably dried, typically by means of distillation or by being passed over an adsorbent.

Use of catalytic compounds: metathesis reactions:

As emphasized above in the present description, the catalytic compositions of the invention are found to be particularly suitable for carrying out the metathesis of unsaturated fatty bodies.

More precisely, the catalytic compositions of the invention are found to be suitable for carrying out homo-metathesis reactions, where two identical molecules of unsaturated fatty body react with each other, and cross-metathesis reactions, where the unsaturated fatty body reacts with a separate olefin compound. The catalyst of the invention may in particular be used as a catalyst for cross-metathesis to carry out ethenolysis reactions of unsaturated fatty bodies (cross-metathesis where the unsaturated fatty body reacts with ethylene, which splits it into two compounds which carry terminal olefin functions>C═CH₂).

The present invention also relates to these specific applications of catalytic compositions. According to a more specific aspect, the invention relates to methods for metathesis of a saturated fatty body with an olefin compound which may or may not be different from this unsaturated fatty body, in which the fatty body is placed in contact with the olefin compound, in the presence of a catalytic composition or a catalyst according to the invention.

In this context, according to a specific embodiment, the invention relates to a method for cross-metathesis of an unsaturated fatty body with an olefin compound which is separate from the fatty body, this olefin compound preferably being used in excess compared with the fatty body. The olefin compound used in this context is typically a linear, terminal or internal olefin. Preferably, this olefin is symmetrical. In particular, the method of cross-metathesis according to the invention may use ethylene, butene-2 or hexene-3 as an olefin compound.

The unsaturated fatty bodies which can be processed according to the cross-metathesis and homo-metathesis reactions of the present invention may vary to a very wide degree.

The metathesis method is found to be particularly suitable for the metathesis of unsaturated fatty bodies comprising at least one carboxylic monoacid which has from 10 to 22 atoms of carbon and which comprises at least one ethylene unsaturation.

The metathesis method of the invention is also suitable for unsaturated fatty acid esters formed from:

at least one monocarboxylic acid which comprises at least one ethylene unsaturation and which advantageously has at least 10 atoms of carbon, for example, at least 12 atoms of carbon; and

at least one saturated aliphatic alcohol (monoalcohol or polyol), for example, a monoalcohol comprising from 1 to 8 atoms of carbon, or a polyol such as glycerol.

More specifically, the method of the invention is found to be generally advantageous when the unsaturated fatty body comprises oleic acid or esters of oleic acid.

The metathesis method of the invention is also found to be suitable for carrying out the metathesis of admixtures comprising the above-mentioned fatty bodies, in particular vegetable oils.

Especially, the metathesis method is suitable for carrying out the metathesis of the unsaturated fatty bodies present in vegetable oils which are rich in oleic acid, in particular oleic sunflower oils, oleic colza oils, and for carrying out metatheses of monoalcohol esters obtained by means of transesterification of these oils. The oleic oils of colza and sunflower are characterized by a composition of fatty acids which is rich in oleic acid (generally at least 80%), the content of linoleic fatty chains does not exceed 12% to the content of linolenic fatty chains does not exceed 0.3% with no other olefin chain being present at a content greater than 0.3% in these oils.

The above-mentioned oils which are rich in oleic acid can be used in their natural form (including triglycerides of fatty acid) or in the form of an admixture of esters of monoalcohol obtained by means of transesterification of the oil (for example, with alcohols comprising from 1 to 8 atoms of carbon, such as methanol, ethanol or propanol).

The metathesis method of the invention also relates to unsaturated fatty bodies which are enriched with monounsaturated fatty acids of the oleic type by means of selective hydrogenation of fatty bodies containing acids which are polyunsaturated at C18.

The metathesis method of the invention, when it is carried out in the presence of ethylene as an olefin compound, allows, from unsaturated fatty acids of the oleic acid type mentioned above, monocarboxylic acids to be obtained which comprise a>C═CH₂ function in the terminal position (ethenolysis of the fatty acid).

In the same manner, starting from monoalcohols of unsaturated fatty acid esters, the method of the invention, when ethylene is present as an olefin compound, leads to monounsaturated monoalcohol esters of monocarboxylic acid comprising a>C═CH₂ function in a terminal position (ethenolysis of the fatty ester).

In a specific manner, the method of the invention in particular allows the ethenolysis of methyl oleate (metathesis reaction from the methyl oleate and ethylene) which allows access to 9-methyl-decenoate.

Regardless of the nature of the unsaturated fatty body used in the metathesis method of the invention, this method is advantageously carried out under the following conditions.

The quantity of catalytic composition used for the metathesis reaction is dependent on a variety of factors, such as the identity of the reagents and reaction conditions used. Consequently, the quantity of catalytic composition required will be defined in an optimal manner and independently for each reaction. Generally, however, the molar ratio of the ruthenium precursor used and the unsaturated fatty body is from 1:50 and 1:10000000. Preferably, the molar ratio of ruthenium/unsaturated fatty body is from 1:500000 and 1:500.

The metathesis reaction of the fatty body according to the invention can be carried out with or without a solvent being present. If necessary, solvents which can be used according to the method of the invention can be selected from organic solvents, protic solvents or water. Solvents which can be used for the metathesis according to the present invention may, for example, be selected from aromatic hydrocarbons (benzene, toluene, xylenes, etc.), halide-containing aromatic hydrocarbons (chlorobenzene, dichlorobenzene, etc.), aliphatic hydrocarbons (pentane, hexane, heptane, cyclohexane, etc.), chlorine-containing alkanes (dichloromethane, chloroform, 1,2-dichloroethane, etc.), ethers (diethylether, tetrahydrofuran, etc.), alcohols (methanol, ethanol, etc.) or water. A very suitable solvent is toluene.

Vigorous agitation is often advantageous in the method of the invention, in so far as it allows good contact from the reagents (which may be gaseous in some cases) and the catalytic admixture.

Furthermore, the implementation temperature of the metathesis reaction of the present invention is typically from −78° C. and 150° C., preferably from 20 and 80° C.

In the case of a gaseous reagent, (such as ethylene, for example), the pressure of the reaction may be from atmospheric pressure and 100 bar (10⁷ Pa), this pressure preferably being from atmospheric pressure and 30 bar (3.10⁶ Pa). This gaseous reagent can be used in the pure state or in admixture or diluted with a paraffin (which is inert).

The reactions of the method of the invention are catalyzed by the catalytic composition which has been described above. The catalytic composition is typically added to the reaction medium as a solid but it can also be added in solution when it is dissolved in an appropriate solvent.

The metathesis reaction of unsaturated fatty bodies according to the invention may be carried out in a closed system (batch), in semi-open systems or in a continuous system, with one or more reaction stage(s).

To further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

Preparation of a catalytic composition C1 according to the invention:

98.8 Preparation of the iminium salt:

An iminium salt complying with the formula below:

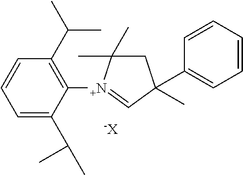

wherein:

X is chloride ions at least partially in the form of HCl₂⁻ was synthesized in accordance with the following reaction:

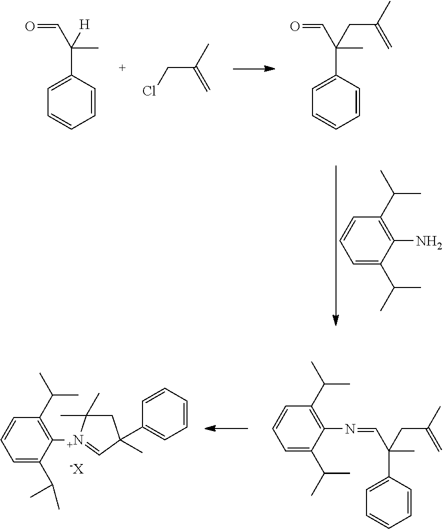

More precisely, the iminium salt was prepared as follows:

Synthesis of 2,4-dimethyl-2-phenyl-pent-4-enal:

A reactor which is provided with mechanical agitation and a heating device (oil bath), was charged with a solution containing 11.76 g (0.13 mole) of 3-chloro-2-methyl-propene and 13.4 g (0.1 mole) of 2-phenyl-propionaldehyde in 50 mL of toluene.

To this solution there was added, drop by drop at a temperature maintained from 70 and 80° C. using the oil bath, an admixture of an aqueous solution at 50% by weight of soda (2 molar equivalents compared with the aldehyde) and tetrabutylammonium bromide (4 molar % compared with the aldehyde). The admixture obtained was then left under agitation for 4 hours 30 minutes at 70-80° C.

Following the reaction, the reaction admixture was cooled to ambient temperature (25° C.), then extracted with 40 mL of distilled water. The aqueous phase obtained was extracted with 3 times 20 mL of toluene and the organic phase obtained during this second extraction was dried over MgSO₄ and filtered over sintered glass No. 4. The filtrate was subjected to reduced pressure (50 mm Hg—Rotary evaporator) to evaporate the solvent, then distilled under reduced pressure.

15 g of the 2,4-dimethyl-2-phenyl-pent-4-enal desired were thus obtained (yield: 80%) in the form of an oil having the following properties:

Boiling temperature: 62-64° C. at 0.8 mbar

NMR¹H d (ppm): 9.49(s, 1 H); 7.25(m, 5H); 4.76(s, 1 H); 4.58(s, 1 H); 2.65 (dd, 2H); 1.42 (s, 3H); 1.36 (s, 3H).

Synthesis of the imine:

The 2,4-dimethyl-2-phenyl-pent-4-enal prepared in the previous step was caused to react with 10 g of 2.6 diisopropyl-phenylamine (0.05 mole) (1.1 equivalent of 2,4-dimethyl-2-phenyl-pent-4-enal for 1 equivalent of 2,6-diisopropyl-phenylamine) in 20 mL of toluene in the presence of a catalytic quantity (2 molar %) of p-toluene sulfonic acid.

The reaction was carried out under reflux of toluene in a single-neck flask upon which there is placed a Dean-Stark and a coolant. The formation of water was observed in the Dean-Stark.

Following the reaction, the toluene was evaporated under reduced pressure (50 mm Hg—Rotary evaporator), then the residue was distilled under reduced pressure.

After purification by means of column chromatography, 10.4 g of the imine were isolated (yield: 65%) having the following characteristics:

NMR¹H d (ppm): 7.66(s,1 H), 7.40(m,2H), 7.30(m,2H), 7.20(m,1 H), 4.79(s,1 H), 4.63(s,1 H), 2.93(m,2H), 2.88(m,2H), 1.65(s,3H), 1.33(s,3H), 1.10(s,12H). IR (cm⁻¹): 1654 (ν C═N), 892 (ν C═C).

Synthesis of the iminium salt (cyclisation):

The imine formed in the preceding step was placed in solution in 30 mL of dry toluene and the solution obtained was cooled to 0° C., then a bubbling of gaseous HCl was established in the solution at 0° C. and maintained for 5 hours.

At the end of these 5 hours of bubbling, the reaction medium was heated to 80° C. for 12 hours. During this step, a color change was observed in the reaction medium but no formation of precipitate.

Following the reaction, the solvent toluene was evaporated to the dry state under reduced pressure (50 mm Hg—Rotary evaporator), resulting in a white powder which was taken up by the ether, filtered over sintered glass No. 4 and dried again.

The iminium salt desired was thus obtained in the form of a white powder (yield: 79%) having the following characteristics:

Melting temperature=204-205° C.

NMR¹H d (ppm): 11.9 (s, 1 H); 7.36 (m, 5H); 7.21 (m, 3H); 3.14 (d, 2H); 2.62 (m, 2H); 1.92 (s, 3H).

1.2 Preparation of the catalytic composition from the iminium salt:

Formation of the carbene from the iminium salt:

To 0.199 g (0.55 mmol) of the iminium salt prepared in step 1.1, there was added slowly, at −78° C., 10 mL of dry THF and 3 molar equivalents of KN(SiMe₃)₂ referred to as KHMDS (0.33 g, 1.65 mmol). The reaction medium thus obtained was placed under agitation at ambient temperature (25° C.) for 16 hours.

After 16 hours of agitation, the solvent present in the reaction medium obtained was evaporated. The residue obtained after evaporation was dissolved in 10 mL of toluene, by which method a solution(s) is/are obtained containing a carbene.

Preparation of the catalytic species by reacting the carbene with a ruthenium compound:

The solution obtained in the preceding step was added, at ambient temperature and under agitation, to 0.55 mmol of a ruthenium compound complying with the following formula:

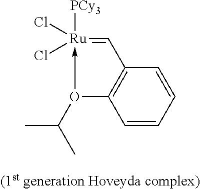

wherein each of the 3 Cy is a cyclohexyl group.

This ruthenium compound is a product which is marketed by ALDRICH under the reference 577944.

The reaction admixture obtained was left under agitation for 16 hours under argon, then evaporated under reduced pressure. The residue obtained was washed by two times 10 mL of pentane, then dried.

A catalyst C1 according to the invention was thus obtained, in the form of a green powder.

This powder, which was characterized by NMR and mass spectroscopy, complies with the following structural formula:

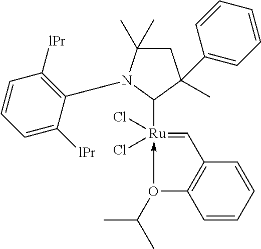

NMR ¹H (300 MHz, C₆D₆): 16.59 (s, 1H, Ru═CH), 8.38 (d, m-CH Ar), 7,5 (m, 2H, p-CH Ar), 7.34-7.23 (m, 4H, CH Ar), 7.08-6.93 (m, 2H, CH Ar), 6.61 (t, 1 H, p-CH), 6.37 (d, 1 H, CH Ar), 4.51 (sept,, 1 H, OCH(CH₃)₂), 3.25 (sept,, 2H, CH(CH₃)₂), 2.87 (d, 1 H, CCH₂), 2.49 (s, 3H, CH₃), 1.93 (d, 1 H, CCH₂), 1.52 (d, 3H, CH₃), 1.35 (d, 3H, CH₃), 1.21 (d, 3H, CH₃), 1.13 (d, 3H, CH₃), 1.05 (m, 9H, CH(CH₃)₃), 0.78 (d, 3H, CH₃)

NMR ¹³C {¹H} (75 MHz, C₆D₆): 293.71; 266.23; 153.53; 149.17; 148.85; 143.54; 143.03; 137.48; 130.53; 130.38; 129.66; 129.09; 126.19; 125.91; 123.9; 121.77; 113.58; 77.13; 74.77; 63.39; 48.96; 29.18 28.53; 28.44; 27.76; 26.73; 24.49; 24.38; 22.42; 22.28

### HRMS (FAB) m/z: 667.1921 [M+]

### EXAMPLE 2

Use of the catalyst C1 of example 1 for the metathesis of an unsaturated fatty body: ethenolysis of the methyl oleate:

Metathesis reactions of (cis) methyl oleate and ethylene catalyzed by variable quantities of the catalyst of example 1 were carried out under different temperature and pressure conditions, in accordance with the protocol below.

The metathesis reaction which takes place in this context is the following cross-metathesis reaction:
CH₃—(CH₂)₇—CH═CH—(CH₂)₇—COOCH₃+H₂C═CH₂
   Methyl oleate
      →CH₃—(CH₂)₇—CH═CH₂+H₂C═CH—(CH₂)₇—COOCH₃
         1-decene 9-methyl-decenoate

In an autoclave of 50 mL, the required quantity of catalyst was introduced as obtained in example 1 (green powder) to obtain a specific concentration of Ru in the medium, which was placed in solution in 1 mL of toluene.

1.05 mL (or 3.46 mmol) of methyl oleate in 20 mL of toluene was then added into the autoclave.

The reactor was then brought to the desired temperature and pressure at time t=0.

Samples were taken over time using a plunger tube to monitor the reaction. Each sample of reaction medium taken in this manner was neutralized using butylvinylether, filtered over a column of celite before being analyzed using gas phase chromatography.

The results obtained after variable reaction times under different conditions of concentration, temperature (T) and pressures in ethylene (P_{C2H2}) used are set out in the tables 1 and 2 below, where the abbreviations used have the following meanings:
OM: methyl oleate
% mol Ru: concentration of ruthenium (reflecting the quantity of catalyst introduced), calculated using the following ratio:

% mol Ru=(n_{Ru})/(n_{OM}ⁱ)×100

wherein n_{Ru} and n_{OM}ⁱ refer, respectively, to the quantities, in mole, of Ru and methyl oleate initially present in the reaction medium.
P_{C2H2}: pressure of ethylene used in the ethenolysis reaction
T: temperature at which the ethenolysis reaction is carried out
t_{R}: duration of the ethenolysis reaction
C_{OM}: conversion of methyl oleate, calculated using the following ratio:

C_{OM}=(n_{OM}ⁱ−n_{OM}^{f})/(n_{OM}ⁱ)×100
where n_{OM}ⁱ and n_{OM}^{f} refer, respectively, to the molar quantities of methyl oleate present initially and at the end of the reaction in the reaction medium.
Salt: selectivity of the ethenolysis reaction carried out, calculated using the ratio below (the formation of 2 moles of ethenolysis product require one mole of reagent OM):

salt=[(n_{1-decene}^{f}+n_{9-methyl decenoate}^{f})/2]/(n_{OM}ⁱ−n_{OM}^{f})×100
where n_{1-decene}^{f} and n_{9-methyl decenoate}^{f} are, respectively, the molar quantities of 1 -decene and 9-methyl decenoate present in the medium following the reaction, and where n_{OM}ⁱ and n_{OM}^{f} have the above-mentioned selectivities.

TABLE 1
Ethenolysis reaction of the methyl oleate using the catalyst prepared
in example 1 - % mol Ru = 0.14% - t_{R} = 2 h:

| Operating conditions | C_{OM} (%) | Salt (%) |
|---|---|---|
| T = 50° C. | 99 | 97 |
| P_{C2H2} = 10⁶ Pa (10 bar) | | |

TABLE 2
Ethenolysis reaction of the methyl oleate using the catalyst prepared
in example 1 - % mol Ru = 0.023% - t_{R} = 2 h:

| Operating conditions | C_{OM} (%) | Salt (%) |
|---|---|---|
| T = 50° C. | 81 | 99 |
| P_{C2H2} = 10⁶ Pa (10 bar) | | |
| T = 23° C. | 16 | >99 |
| P_{C2H2} = 10⁶ Pa (10 bar) | | |
| T = 50° C. | 54 | >99 |
| P_{C2H2} = 3.10⁶ Pa (30 bar) | | |
| T = 50° C. | 47 | 99 |
| P_{C2H2} = 1.5 × 10⁵ Pa (1.5 bar) | | |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following Claims, including equivalents thereof.

What is claimed is:

1. A catalytic composition which comprises a ruthenium-based catalyst having the formula (I) below: 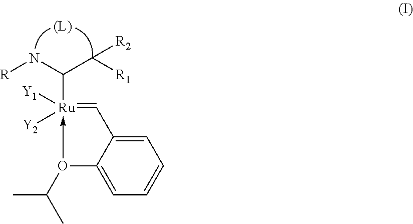
wherein:
Y₁ and Y₂ are two anionic ligands which may be identical or different;
R is a branched alkyl radical, an aryl radical or a substituted aryl radical;
R₁ is a group comprising an aromatic radical;
R₂ is an aryl, alkylaryl or arylalkyl radical which may or may not be substituted and which is identical or different to R₁; or an alkyl radical; and
the group -(L)- is a divalent hydrocarbon radical, with the proviso that the ring having the following formula: 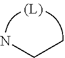
formed by this divalent group -(L)-, nitrogen, the carbon linked to the ruthenium and the carrier carbon of the radicals R₁ and R₂, is a ring having 5 or 6 atoms, the nitrogen atom included.

2. The catalytic composition as defined by claim 1, wherein, in the compound (I), R₁ is an aryl, alkylaryl or arylalkyl radical, whether or not substituted.

3. The catalytic composition as defined by claim 1, wherein, in the catalyst having the formula (I), the two ligands Y₁ and Y₂ are chlorides.

4. The catalytic composition as defined by claim 1, wherein, in the catalyst having the formula (I), the divalent radical -(L)- has the following formula: 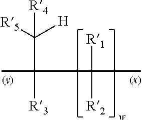
wherein
(x) and (y) indicate the two bonds which are established from the carbon atom which carries the radicals R₁ and R₂ and the nitrogen atom which carries the radical R;
w is a number equal to 1 or 2;
each of the radicals R′₁, R′₂, R′₃, R′₄ and R′₅, which may be identical or different, is an atom of hydrogen or an alkyl, cycloalkyl, aryl or arylalkyl radical.

5. The catalytic composition as defined by claim 1, wherein the catalyst has the following formula (II): 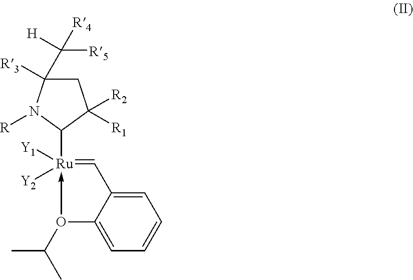
wherein:
R is a branched alkyl radical, an aryl radical, or a substituted aryl radical;
R₁ is an aryl or substituted aryl radical;
R₂ is an aryl radical or a substituted aryl radical identical or different to R₁; or an alkyl radical;
R′₃ is a hydrogen atom, an alkyl radical, an aryl radical, or a substituted aryl radical;
each of R′₄ and R′₅, which may be identical or different, is an atom of hydrogen, an alkyl radical, an aryl radical, or a substituted aryl radical.

6. The catalytic composition as defined by claim 5, wherein:
R is a tert-butyl radical; a phenyl radical which is substituted by 3 methyl or ethyl groups in the position o, o′ and p; a phenyl radical which is substituted by 2 isopropyl or tert-butyl radicals in the position o and o′; a phenyl radical which is substituted by 3 isopropyl or tert-butyl radicals in the position o, o′ and p; or a phenyl radical which is substituted by two ethyl groups in the positions o and o′;
R₁ is a phenyl, substituted phenyl, naphthyl or substituted naphthyl radical;
R₂ is a phenyl, substituted phenyl, naphthyl or substituted naphthyl radical, which may be identical or different to R₁, or a methyl, ethyl, propyl, or isopropyl radical;
R′₃ is an atom of hydrogen or a methyl radical, and
each of the radicals R′₄, R′₅, which may be identical or different, is an atom of hydrogen or a methyl radical.

7. The catalytic composition as defined by claim 5, wherein:
R is a phenyl radical which is substituted by two isopropyl groups in the position o and o′; a phenyl radical which is substituted by 3 methyl or isopropyl radicals in the position o, o′ and p; or a phenyl radical which is substituted by two ethyl groups in the positions o and o′ or a tert-butyl radical;
R₁, R₂, which may be identical or different, are each a phenyl, substituted phenyl, naphthyl or substituted naphthyl radical;
R′₃ is a methyl radical, and
R′₄ and R′₅ are both atoms of hydrogen.

8. The catalytic composition as defined by claim 1, wherein the catalyst has the formula (III) below: 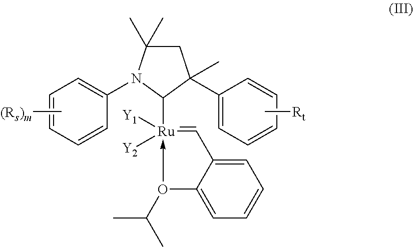
wherein:
m is the number of substituents carried by the aryl radical which is bonded to the nitrogen, this number being equal to 1, 2 or 3, each of the m radicals Rₛ, which may be identical or different, is a substituent selected from among the alkyl or alkoxy radicals which have from 1 to 4 atoms of carbon,
Rₜ is one or more substituents, which may be identical or different, selected from among the alkyl or alkoxy radicals which have from 1 to 4 atoms of carbon.

9. A catalyst having the formula (I) below: 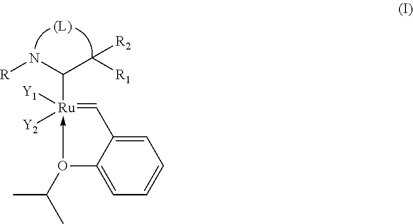
wherein:
Y₁, Y₂, R, R₁, R₂ and -(L)- are as defined claim 1.

10. The catalyst having either formula (II) or formula (III) below: 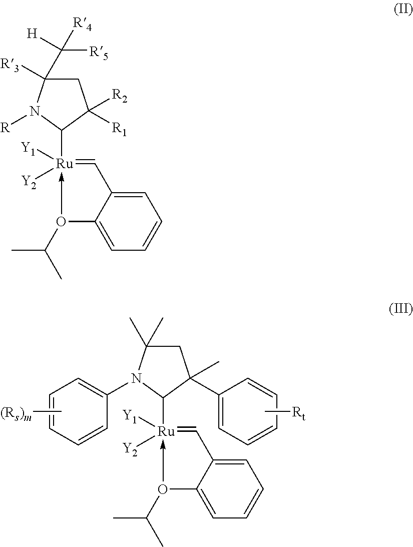
wherein:
R, R₁, R₂, R′₃, R′₄, R′₅, Y₁ and Y₂ are as defined in claim 5, m is 1, 2 or 3 and t is the number of substiuents R.

11. A method for preparing a catalytic composition as defined by claim 1, comprising contacting:
a ruthenium-based precursor having the general formula (IV) below: 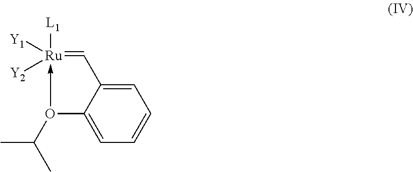
wherein:
Y₁ and Y₂ are ligands, which may be identical or different; and
L₁ is a neutral electron-donating ligand; and
a carbene having the formula (V) below:

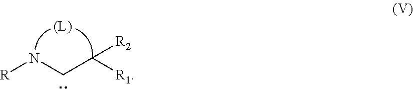

12. The method as defined by claim 11, wherein, in the ruthenium based precursor (IV), the ligand L₁ is a phosphine group.

13. The method as defined by claim 12, wherein the ruthenium-based precursor has the formula (IVa) below: 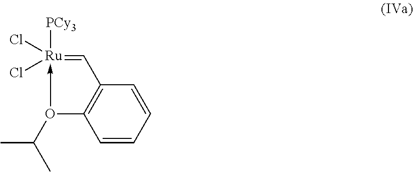
wherein Cy is a cyclohexyl radical.

14. The method as defined by claim 11, wherein the carbene (V) is obtained by reacting:
an iminium salt having the formula (VI) below: 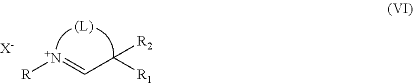
wherein X⁻ is an anion, with a basic, organic or inorganic compound.

15. The method as defined by claim 14, wherein X⁻ is a chloride anion in the form Cl⁻ and/or HCl₂⁻.

16. The method as defined by claim 14, which comprises the following steps:
(E1) at least one iminium salt having the formula (VI) is contacted with at least one base, which may be organic or inorganic, whereby a carbene having the formula (V) is formed;
(E2) optionally, the carbene (V) formed during step (E1) is isolated;
(E3) the ruthenium-based precursor having the formula (IV) is added to the carbene obtained in step (E1) and is optionally isolated beforehand in step (E2), whereby the catalyst having the formula (I) is formed;
(E4) optionally, the catalyst formed in step (E3) is isolated,
(E5) optionally, a solvent is added to the catalyst formed in step E3, optionally isolated beforehand in step (E4).

17. A catalytic composition as prepared by the method as defined by claim 16.

18. A method for metathesis of an unsaturated fatty body, wherein the unsaturated fatty body is contacted with an olefin compound which may or may not be different from the unsaturated fatty body, in the presence of a catalytic composition as defined by claim 1.

19. The method as defined by claim 18, comprising cross-metathesis of the unsaturated fatty body, wherein the olefin compound is a linear olefin which is different from the fatty body.

20. The method as defined by claim 19, wherein the olefin compound is a symmetrical linear olefin selected from among ethylene, butene-2 or hexene-3.

21. The method as defined by claim 20, wherein the olefin compound is ethylene.

22. The method as defined by claim 18, wherein the unsaturated fatty body comprises at least one carboxylic monoacid which has from 10 to 22 atoms of carbon and which comprises at least one site of ethylene unsaturation or unsaturated fatty acid esters formed from:
at least one monocarboxylic acid which comprises at least one ethylene unsaturation and which has at least 10 atoms of carbon; and
at least one saturated aliphatic alcohol (monoalcohol or polyol), a monoalcohol comprising from 1 to 8 atoms of carbon, or a polyol.

23. The method as defined by claim 21, wherein the unsaturated fatty body comprises oleic acid or esters of oleic acid.

24. The method as defined by claim 23, wherein the unsaturated fatty body is methyl oleate and wherein the olefin compound is ethylene, and forming 9-methyl decenoate by ethenolysis of the methyl oleate.

25. A method for metathesis of an unsaturated fatty body, wherein the unsaturated fatty body is contacted with an olefin compound which may or may not be different from the unsaturated fatty body, in the presence of a catalytic composition as defined by claim 8.
